(19) United States
(12) Patent Application Publication
(10) Pub. No.: US 2009/0291137 A1
(43) Pub. Date: Nov. 26, 2009

(54) Solid oral form provided with a double release profile

(75) Inventors: Florence Guimberteau, Montussan (FR); Anne-Sophie Daviaud, Saint Genis Laval (FR)

Correspondence Address: PATTON BOGGS LLP, 8484 WESTPARK DRIVE, SUITE 900, MCLEAN, VA, 22102, US

(73) Assignee: Flamel Technologies, S.A., Venissieux Cedex (FR)

(21) Appl. No.: 12/425,875

(22) Filed: Apr. 17, 2009

Related U.S. Application Data
(60) Provisional application No. 61/071,250, filed on Apr. 18, 2008.

(30) Foreign Application Priority Data
Apr. 18, 2008 (FR) 08 52627
Apr. 17, 2009 (FR) PCT/FR2009/050719

(51) Int. Cl. A61K 9/26; A61K 9/14

(52) U.S. Cl. 424/469; 424/497; 424/484

(57) ABSTRACT

The present invention relates to a solid form, intended for the administration by oral route of at least one active ingredient and capable of guaranteeing a double release mechanism of said active ingredient, the first being determined by time and the second being determined by the pH, characterized in that said active ingredient is present there in the form of a microparticle system the microparticles of which possess a core formed wholly or partly by said active ingredient and coated with at least one layer determining said release profile of said active ingredient and formed by a material composed at least (i) 25 to 75% by weight relative to the total weight of said coating of at least one polymer A which is insoluble in the gastro-intestinal fluids, (ii) 25 to 75% by weight relative to the total weight of said coating of at least one polymer B possessing a solubilization pH value comprised within the pH range from 5 to 7, and (iii) 0 to 25% by weight relative to the total weight of said coating of at least one plasticizer, said polymers A and B being present in a polymer(s) B/polymer(s) A weight ratio at least equal to 0.25

It moreover relates to a method for the preparation of this solid form and of the corresponding microparticles.

The present invention aims to propose a solid or also tablet form, intended for administration by oral route, containing at least one active ingredient formulated in the state of microparticles, said microparticles in free form and said final solid form containing them being provided with the same specific modified release profile.

The present invention also refers to a useful method for the preparation of such a solid form.

The prior art has shown the usefulness of having multiparticle oral pharmaceutical forms. These microparticle systems are constituted by a large number of microcapsules or microparticles with a diameter generally less than 2000 μm. These systems are advantageous on several counts.

First of all, the dose of active ingredient(s) to be administered is to be found therein distributed between a large number of microparticles, typically 10,000 for a dose of 500 mg, and therefore exhibits a low sensitivity to the variability of gastric emptying and virtually zero risk of tissues being brought into contact with a high dose of active ingredient(s).

Moreover, the microparticle systems allow the utilization, within a single dose unit such as a gelatin capsule for example, of a mixture of microparticles with different modified release profiles, thus making it possible to produce release profiles having several waves of release or ensuring, by a suitable regulation of the different proportions, a constant concentration level of active ingredient(s) in the plasma.

By way of illustration of these modified release forms in the multiparticle state, there can in particular be mentioned those described in the documents US 2002/0192285, U.S. Pat. No. 6,238,703, US 2002/0192285, US 2005/0118268 and U.S. Pat. No. 5,800,836 and in particular those described in the Application WO 03/030878.

Thus the application WO 03/02078 describes more particularly microparticle oral pharmaceutical forms allowing a release of the active ingredient that they contain according to a double modified release mechanism, the first being determined by time and the second being determined by the pH. More precisely, this release process can be schematically represented by a sequence of three distinct phases: a first so-called latency phase followed by a second so-called controlled release phase, which are both manifested on contact with an acid medium representative of the gastric medium, followed by a third so-called accelerated or even immediate release phase, which is manifested on contact with a neutral medium representative of the intestinal medium.

This multiparticle system thus allows a modified, delayed and sustained release of the active ingredient, the different sequences of which are triggered according to two distinct mechanisms respectively activated by time and by the pH. Moving from the first phase to the second phase is triggered by a time of contact with the acid medium representative of the gastric medium, whereas moving from the second phase to the third phase is triggered by the change of pH encountered when the microparticles leave the stomach to enter the intestine.

This particular type of modified release profile is particularly useful in the following cases:
when a delayed release is sought, either to adapt the release of the active ingredient to a chronobiological cycle whilst maintaining an administration schedule compatible with everyday life, or to delay the release of one active ingredient with respect to another within a combination; and
when the active ingredient considered is highly metabolized by the liver without the metabolites being active. A modified release in the form of several shifted peaks in this case makes it possible to minimize the hepatic metabolism and to retain bioavailability whilst prolonging the period of action of the active ingredient.

In all these cases, the formulations having the specific 3-phase release profile are superior in terms of variability to the formulations commonly called enteric and generally used to obtain a delayed release.

The conventional enteric formulations in fact have only 2 phases: a phase of non-release or also of latency in acid medium representative of the gastric medium and a phase of immediate release in neutral medium representative of the intestinal medium. In the case of these standard enteric forms, the release of the active ingredient is triggered by the change in pH linked to the form moving from the stomach into the intestine. Now, this movement is extremely variable from one individual to another, and even from one moment to another in the same individual. It is not unusual for an oral form to be retained in the stomach for much longer than expected, up to 18 hours for example. Thus, for a product administered every 24 hours, if the first tablet is retained for 18 hours and if the following tablet passes into the intestine much more rapidly, the patient will not be treated on the first day, but will receive the equivalent of 2 doses on the second day. This variability can have negative consequences if the active ingredient has a low therapeutic index, i.e. if a high plasma level of this active ingredient is associated with serious side effects.

The formulations having a specific three-phase release profile avoid this problem and make it possible to access delayed and sustained release profiles with a low and acceptable variability, even for active ingredients with a low therapeutic index.

They are generally presented in the form of microparticles or microcapsules the core of which, containing the active ingredient or a mixture of active ingredients, is covered with a coating the composition and/or thickness of which are precisely adjusted in order to control the release of this active ingredient according to two distinct mechanisms, depending on whether the coated core is located in the stomach or the small intestine, the one being determined by the residence time in an acid aqueous medium and the other by the pH of the medium containing the microparticles.

For example, the coating of the microparticles described in WO 03/03878 is formed by a material comprising at least one hydrophilic polymer bearing groups ionized at neutral pH, such as for example a (meth)acrylic acid and alkyl (meth) acrylate copolymer and at least one hydrophobic compound in the form of a hydrogenated vegetable wax. Such microparticles are completely satisfactory in terms of modified release profile, when they are formulated in a non-compressed pharmaceutical system such as a powder or a gelatin capsule.

Unfortunately, the formulation of this type of microparticles in a compressed oral solid form such as a tablet generally proves prejudicial to the modified release profile. In particular, the initial latency time is generally lost under the effect of the accelerated release of the active ingredient of at least some of the microparticles, the coating of which has been broken by the compression force, applied during the formulation of the tablet.

Now, among all the solid forms, the compacted or also cohesive solid forms, in the form of tablets, are advantageous on several counts.

Unlike powders, they do not require pre-dilution in an aqueous medium and therefore can be ingested instantaneously by the patient with, moreover, a total guarantee of the dosage of active ingredient received by the patient. Moreover, their industrial production is clearly less restrictive for the formulator compared with gelatin capsules or capsules. Furthermore, compared with the latter, the solid forms of tablet type possess a better mechanical strength. They are not friable whilst being compatible with fragmentation into several dosage parts if necessary (scored tablets).

A need therefore remains for a solid formulation presented in tablet form and constituted by a large number of microparticles provided with a specific three-phase release profile for the active ingredient carried within this formulation.

In particular, a need remains for a compressed solid form constituted by modified three-phase release microparticles such that the final compressed form has the same modified three-phase release profile as the modified release microparticles that it contains, considered in free form.

In particular, a need remains for a solid formulation capable of providing a modified three-phase release profile, resulting from a double release mechanism, the first being determined by time, to the extent that the release of the active ingredient is triggered after a determined residence time of the solid formulation in the stomach and the second being determined by the pH, to the extent that the release of the active ingredient is accelerated once the solid formulation is brought into contact with the medium contained in the small intestine.

Unexpectedly, the inventors have found that it is possible to have solid oral forms available, produced by compression, which are nevertheless capable of guaranteeing such a modified three-phase active ingredient(s) release profile, providing that, within these solid forms, the active ingredient is dispersed in the form of microparticles provided with a specific coating.

More precisely, the present invention, according to a first aspect, relates to a solid form intended for the administration by oral route of at least one active ingredient and capable of guaranteeing a double release mechanism of said active ingredient, the first being determined by time and the second being determined by the pH, characterized in that said active ingredient is present there in the form of a microparticle system the microparticles of which possess a core formed wholly or partly by said active ingredient and coated with at least one layer determining said modified release profile of said active ingredient and formed by a material made up of at least:
10 to 75% by weight and in particular 25 to 75% by weight relative to the total weight of said coating of at least one polymer A which is insoluble in the gastro-intestinal fluids,
25 to 90% by weight and in particular 25 to 75% by weight relative to the total weight of said coating of at least one polymer B having a solubilization pH value varying within the pH range from 5 to 7 and
0 to 25% by weight relative to the total weight of said coating of at least one plasticizer,
   said polymers A and B being present in a polymer(s) B/polymer(s) A weight ratio at least equal to 0.25.

The expression “double release mechanism of said active ingredient, the first being determined by time and the second being determined by the pH” can also be denoted in simplified manner by the term “double release mechanism in terms of time and pH”.

Within the meaning of the invention, the term “oral solid form” generally denotes tablets intended for administration by oral route.

The expression “double release mechanism” expresses the fact that the microparticles have two distinct release mechanisms for the active ingredient which can also be schematically represented in the form of a three-phase release profile:
a first release mechanism deferred in time on contact with an acid medium. This release mechanism can be split into a first latency phase followed by a second controlled release phase. In other words, the oral solid forms according to the invention possess an ability to initiate the release of the active ingredient that they contain in acid aqueous medium, only after at least 30 minutes in contact with this medium
a second accelerated or even immediate release mechanism on contact with a neutral aqueous medium. This second release mechanism can be schematically represented by a third release phase.

Therefore, the solid form considered according to the invention is capable, on the one hand, of releasing in a sustained fashion the active ingredient that it contains after a latency period, determined by a given residence time in the stomach and, on the other hand, of triggering an accelerated release of the active ingredient on entry of the solid form into the intestine where it is confronted with an increase in pH. These two release mechanisms of the active ingredient or ingredients formulated in the solid form according to the invention are ensured in sequence.

Within the meaning of the present invention, the pH value for solubilization of the polymer B is a pH value of the physiological medium or of the model in vitro medium below which the polymer is insoluble and above which this same polymer B is soluble.

For obvious reasons, this pH value is specific to a given polymer and directly linked to its intrinsic physico-chemical characteristics, such as its chemical nature and its chain length.

Within the meaning of the present invention, a solid form is a solid form provided with a mechanical breaking strength. It is also advantageously non-deformable.

In view of these specificities, it differs from the pharmaceutical forms also qualified as “solid”, such as for example gelatin capsules and powders.

Advantageously, a solid form according to the invention is presented in the form of a matrix in which the microparticles containing the active ingredient or the mixture of active ingredients to be carried are dispersed.

More precisely, they are obtained by the compression of the different compounds and/or materials used in their composition.

According to a preferred embodiment, the solid form according to the invention is a form of tablet type.

According to a preferred embodiment, the solid form possesses a hardness varying from 50 to 500 N.

According to an embodiment variant, a solid form according to the invention can contain at least two types of microparticles, said types differing from each other at least by the nature of the active ingredient that they contain and/or by the composition and/or the thickness of the coating forming their respective particles.

According to another embodiment variant, the solid composition according to the invention can comprise at least two types of microparticles, differing from each other by distinct release profiles.

According to yet another embodiment variant, a solid form according to the invention can contain, apart from the particles possessing a double release mechanism as defined previously, particles provided with a profile for the immediate release of the active ingredient or ingredients that they contain.

According to yet another of its aspects, the present invention relates to a method for producing a solid form according to the invention, as defined hereafter.

Finally, according to another of its aspects, the present invention relates to specific microparticles as defined hereafter.

Solid Form

As specified previously, a solid form according to the invention is advantageously produced by compression. It can of course also be subjected to complementary treatments, in particular as defined hereafter.

Given this method of preparation, it has a significant breaking strength. For example, for a round tablet with a diameter of 12 mm, this hardness can vary from 50 to 500 N, in particular from 60 to 200 N.

This hardness can be measured according to the protocol described in the European Pharmacopoeia 6th Edition, Chapter 2.9.8.

Unexpectedly, this mechanical cohesion does not moreover prove prejudicial to the demonstration, by the microparticles dispersed in said solid composition, of the specific modified three-phase release profile of the active ingredient(s) that they carry.

The microparticles, when they are released from the matrix forming the solid form according to the invention, generally by disintegration of the latter on contact with an aqueous medium, remain capable, thanks to the specific composition of their coating, of releasing the active ingredient according to a specific modified three-phase release profile, as described previously, within the gastro-intestinal tract.

More precisely, when these microparticles are placed in a medium the pH of which is at a value less than that of the solubilization pH of the polymer B forming said particles, a delayed and sustained release profile is observed with a given latency period comprised between 0.5 and 12 hours, in particular between 0.5 and 8 hours, or even between 1 and 5 hours and according to a half release time t_{1/2} comprised between 0.75 and 24 hours, in particular between 0.75 and 12 hours, or even between 0.75 and 8 hours, in particular between 1 and 5 hours, a time at the end of which half of the active ingredient content is released.

On the other hand, when these same microparticles, having previously remained in the stomach or in a comparable medium i.e. at a pH lower than the solubilization pH of the polymer B, are brought into the presence of a medium the pH of which is at a value greater than that of the solubilization pH of the polymer B forming said particles, a release of the active ingredient is observed with no latency period and with a t_{1/2}, comprised between 0.1 and 10 hours, in particular between 0.1 and 5 hours, in particular between 0.1 and 2 hours.

The latency period corresponds to the time below which the microparticles release less than 20% of their dose of active ingredient(s).

Microparticle System

The invention comprises microparticles the composition and architecture of which are adjusted in order to confer precisely the sought modified release profile for the active ingredient or mixture of active ingredients that they contain.

More precisely, the microparticles considered according to the invention are structurally organized in a core, wholly or partly formed by at least one active ingredient or mixture of active ingredients, and coated or film-coated with a coating.

This core can be:

raw (pure) active ingredient, and/or

a matrix granulate containing the active ingredient or a mixture of active ingredient(s) mixed with other, different ingredients and/or
a granulate obtained by the application of a layer formed wholly or partly by the active ingredient onto a support particle, for example constituted by cellulose or sugar.

In the case of a matrix granulate, the matrix can contain the active ingredient and optionally other physiologically acceptable excipients, such as binding agents, surfactants, disintegrators, fillers, agents controlling or modifying the pH (buffers).

In the case of the use of a support particle, the latter can be composed of saccharose and/or dextrose and/or lactose, and/or a saccharose/starch mixture. It can also be a microsphere of cellulose or any other particle of physiologically acceptable excipient. Advantageously, the support particle has an average diameter of less than 1500 μm and preferably comprised between 20 and 1000 μm, preferably between 50 and 1000 μm, in particular between 50 and 800 μm, or even between 50 and 600 μm. The active layer can optionally comprise, apart from the active ingredient(s), one or more physiologically acceptable excipients, such as binding agents, surfactants, disintegrators, fillers, agents controlling or modifying the pH (buffers).

According to a particular embodiment variant, the core forming the microparticles is a granulate obtained by the application of a layer formed wholly or partly by the active ingredient onto a support particle as defined above.

In the case of the present invention, the coating possesses a composition adjusted in order to provide the specific release profile of the active ingredient or associated mixture of active ingredients, i.e. in three phases triggered by a double release mechanism, activated by time and pH.

More precisely, the coating is formed by a composite material produced by mixing:
at least one polymer A which is insoluble in the liquids of the digestive tract;
at least one second polymer B possessing a solubilization pH value comprised within the pH range from 5 to 7;
and optionally at least one plasticizer and/or other conventional excipients.

Polymer A

This polymer which is insoluble in the liquids of the digestive tract or also the gastro-intestinal fluids is more particularly selected from:
non-water-soluble cellulose derivatives,
non-water-soluble (meth)acrylic (co)polymer derivatives,
and mixtures thereof.

More preferably, it can be chosen from ethylcellulose, for example those marketed under the name Ethocel®, cellulose acetate butyrate, cellulose acetate, ammonio (meth)acrylate copolymers (ethyl acrylate, methyl methacrylate and trimethylammonio ethyl methacrylate copolymer) of type “A” or of type “B” in particular those marketed under the names Eudragit® RL and Eudragit® RS, poly(meth)acrylic acid esters, in particular those marketed under the name Eudragit® NE and mixtures thereof.

Ethylcellulose, cellulose acetate butyrate and the ammonio (meth)acrylate copolymers in particular those marketed under the name Eudragit RS® and Eudragit RL® are quite particularly suitable for the invention.

The coating of the microparticles contains 10% to 75%, and can preferably contain 15% to 60%, more preferably 20% to 55%, in particular 25% to 55% by weight, and still more particularly 30 to 50% polymer(s) A relative to its total weight.

According to an embodiment variant, the coating of the particles contains 35% to 65%, preferably 40% to 60% by weight polymer(s) A relative to its total weight.

Polymer B

By way of non-limitative illustration of the polymers (B) suitable for the invention, there can in particular be mentioned:
the methacrylic acid and methyl methacrylate copolymer(s),
the methacrylic acid and ethyl acrylate copolymer(s),
cellulosic derivatives such as:
   cellulose acetate phthalate (CAP),
   cellulose acetate succinate (CAS),
   cellulose acetate trimellitate (CAT),
   hydroxypropylmethylcellulose phthalate (or hypromellose phthalate) (HPMCP),
   hydroxypropylmethylcellulose acetate succinate (or hypromellose acetate succinate) (HPMCAS),
shellac gum,
polyvinyl acetate phthalate (PVAP),
and mixtures thereof.

According to a preferred embodiment of the invention, this polymer B is chosen from the methacrylic acid and methyl methacrylate copolymer(s), the methacrylic acid and ethyl acrylate copolymer(s) and mixtures thereof.

As specified previously, the polymer B considered according to the invention possesses a different solubility profile depending on whether it comes into contact with a pH value above or below its solubilization pH value.

Within the meaning of the invention, the polymer B is generally insoluble at a pH value below its solubilization pH value and by contrast soluble at a pH value above its solubilization pH value.

For example, it can be a polymer the solubilization pH value of which is:
5.0 such as for example hydroxypropylmethylcellulose phthalate and in particular that marketed under the name HP-50 by Shin-Etsu,
5.5 such as for example hydroxypropylmethylcellulose phthalate and in particular that marketed under the name HP-55 by Shin-Etsu or methacrylic acid and ethyl acrylate copolymer 1:1 and in particular that marketed under the name Eudragit L100-55 by Evonik,
6.0 such as for example a methacrylic acid and methyl methacrylate copolymer 1:1 and in particular that marketed under the name Eudragit L100 by Evonik,
7.0 such as for example a methacrylic acid and methyl methacrylate copolymer 1:2 and in particular that marketed under the name Eudragit S100 by Evonik.

All of these polymers are soluble at a pH value above their solubilization pH.

The coating is advantageously made up of at least 25 to 90%, in particular 30 to 80%, more particularly 30 to 75%, in particular 35 to 70%, in particular 35 to 65%, or even 40 to 60% by weight polymer(s) B relative to its total weight.

Advantageously, the coating is formed by a mixture of the two categories of polymers A and B in a polymer(s) B/polymer(s) A weight ratio greater than 0.25, in particular greater than or equal to 0.3, in particular greater than or equal to 0.4, in particular greater than or equal to 0.5, or even greater than or equal to 0.75.

According to another embodiment variant, the polymer(s) B/polymer(s) A ratio is moreover less than 8, in particular less than 4, or even less than 2 and more particularly less than 1.5.

By way of examples representative of the polymer A and B mixtures which are quite particularly suitable for the invention, there can in particular be mentioned the mixtures of ethylcellulose, cellulose acetate butyrate or ammonio (meth)acrylate copolymer of type A or B with at least one methacrylic acid and ethyl acrylate copolymer or a methacrylic acid and methyl methacrylate copolymer or a mixture thereof.

Thus, according to a particular embodiment, the particles according to the invention can be advantageously formed by at least one polymer B/polymer A pair chosen from the following pairs:
1. methacrylic acid and ethyl acrylate, 1:1 copolymer/ethylcellulose,
2. methacrylic acid and methyl methacrylate 1:2 copolymer/ethylcellulose,
3. mixture of methacrylic acid and ethyl acrylate 1:1 copolymer and methacrylic acid and methyl methacrylate, 1:2 copolymer/ethylcellulose,
4. methacrylic acid and ethyl acrylate 1:1 copolymer/cellulose acetate butyrate,
5. methacrylic acid and methyl methacrylate 1:2 copolymer/cellulose acetate butyrate,
6. mixture of methacrylic acid and ethyl acrylate copolymer 1:1 and of methacrylic acid and methyl methacrylate 1:2 copolymer/cellulose acetate butyrate,
7. methacrylic acid and ethyl acrylate 1:1 copolymer/type “A” or type “B” ammonio (meth)acrylate copolymer,
8. methacrylic acid and methyl methacrylate 1:2 copolymer/type “A” or type “B” ammonio (meth)acrylate copolymer,
9. mixture of methacrylic acid and ethyl acrylate 1:1 copolymer and methacrylic acid and methyl methacrylate 1:2 copolymer/type “A” or type “B” ammonio (meth)acrylate copolymer.

According to another of its aspects, a subject of the present invention is microparticles possessing a core formed wholly or partly by at least one active ingredient, said core being coated with at least one layer determining a double release mechanism, the first being determined by time and the second being determined by the pH of said active ingredient and formed by a material made up of at least:
10 to 75% by weight, and in particular 25 to 75% by weight, in particular 25% to 60% and, still more preferably, 25% to 55%, and still more particularly 30 to 50% relative to the total weight of said coating of at least one polymer A which is insoluble in the gastro-intestinal fluids, chosen from ethylcellulose, cellulose acetate butyrate, a type “A” or type “B” ammonio (meth)acrylate copolymer, poly(meth)acrylic acid esters and mixtures thereof, and
25 to 90% by weight, and in particular 25 to 75% by weight, in particular 30 to 75%, in particular 35 to 70%, or even 40 to 60% by weight relative to the total weight of said coating of at least one polymer B possessing a solubilization pH value comprised within the pH range varying from 5 to 7 and chosen from a methacrylic acid and methyl methacrylate copolymer, a methacrylic acid and ethyl acrylate copolymer and mixtures thereof.

Advantageously, the coating can be formed by a polymer B/polymer A pair chosen from the abovementioned pairs.

Apart from the abovementioned two types of polymers, the coating of the particles according to the invention can comprise at least one plasticizer.

Plasticizer

This plasticizer can in particular be chosen from:
glycerol and its esters, and preferably from the acetylated glycerides, glyceryl-monostearate, glyceryl-triacetate, glyceryl-tributyrate,
the phthalates, and preferably from dibutylphthalate, diethylphthalate, dimethylphthalate, dioctylphthalate,
the citrates, and preferably from acetyltributylcitrate, acetyltriethylcitrate, tributylcitrate, triethylcitrate,
the sebacates, and preferably from diethylsebacate, dibutylsebacate,
the adipates,
the azelates,
the benzoates,
chlorobutanol,
the polyethylene glycols,
vegetable oils,
the fumarates, preferably diethylfumarate,
the malates, preferably diethylmalate,
the oxalates, preferably diethyloxalate,
the succinates; preferably dibutylsuccinate,
the butyrates,
the cetyl alcohol esters,
the malonates, preferably diethylmalonate,
castor oil,
and mixtures thereof.

In particular, the coating can comprise less than 25% by weight, preferably 1% to 20% by weight, and, still more preferably, 5% to 20%, in particular 5% to 15% and still more preferably approximately 10% by weight plasticizer(s) relative to its total weight.

Thus, the coating of particles according to the invention can be advantageously formed of at least
20 to 60%, in particular 30 to 60% by weight by at least one polymer A chosen from ethylcellulose, cellulose acetate butyrate, a type “A” or type “B” ammonio (meth)acrylate copolymer or a mixture thereof,
30 to 70%, in particular 40 to 70% by weight at least one polymer B chosen from a methacrylic acid and methyl methacrylate copolymer, in particular a methacrylic acid and methyl methacrylate copolymer 1:1 or a methacrylic acid and methyl methacrylate copolymer 1:2; a methacrylic acid and ethyl acrylate copolymer, in particular a methacrylic acid and ethyl acrylate copolymer 1:1 or a methacrylic acid and ethyl acrylate copolymer 1:2, and mixtures thereof
and approximately 10% by weight at least one plasticizer such as for example triethylcitrate.

As a non-limitative illustration of the particles according to the invention, there can in particular be mentioned those the coating of which possesses one of the following compositions.
30 to 60% ethylcellulose
   40 to 70% methacrylic acid and ethyl acrylate copolymer 1:1
   10% triethylcitrate
30 to 60% ethylcellulose
   40 to 70% methacrylic acid and methyl methacrylate copolymer 1:2
   10% triethylcitrate
30 to 60% ethylcellulose
   40 to 70% a mixture of methacrylic acid and ethyl acrylate copolymer 1:1 and methacrylic acid and methyl methacrylate copolymer 1:2
   10% triethylcitrate
30 to 60% cellulose acetate butyrate
   40 to 70% methacrylic acid and ethyl acrylate copolymer 1:1
   10% triethylcitrate
30 to 60% cellulose acetate butyrate
   40 to 70% methacrylic acid and methyl methacrylate copolymer 1:2
   10% triethylcitrate
30 to 60% cellulose acetate butyrate
   40 to 70% a mixture of methacrylic acid and ethyl acrylate copolymer 1:1 and methacrylic acid and methyl methacrylate copolymer 1:2
   10% triethylcitrate
30 to 60% type “A” or type “B” ammonio (meth)acrylate copolymer
   40 to 70% methacrylic acid and ethyl acrylate copolymer 1:1
   10% triethylcitrate
30 to 60% type “A” or type “B” ammonio (meth)acrylate copolymer
   40 to 70% methacrylic acid and methyl methacrylate copolymer 1:2
   10% triethylcitrate and
30 to 60% type “A” or type “B” ammonio (meth)acrylate copolymer
   40 to 70% a mixture of methacrylic acid and ethyl acrylate copolymer 1:1 and methacrylic acid and methyl methacrylate copolymer 1:2
   10% triethylcitrate

Of course, the coating can comprise various other additional adjuvants used in a standard fashion in the field of coating. It can, for example, comprise pigments, colorants, fillers, anti-foaming agents, surfactants etc.

According to a particular embodiment of the invention, the coating contains no active ingredient.

According to another embodiment of the invention, the coating is devoid of compound soluble at a pH value ranging from 1 to 4.

The coating can be single or multi-layer. According to a variant embodiment, it is made up of a single layer formed by the composite material defined previously.

The coating of the microparticles, whether they are in the free state or dispersed within a solid composition according to the invention, advantageously possesses the same appearance. It is preferably presented in the form of a continuous film arranged on the surface of the core formed wholly or partly by the active ingredient. It advantageously possesses a mechanical strength sufficient to be compatible with exposure to a significant compression force for example of at least 5 kN, in particular of at least 7 kN and preferably greater than 10 kN.

This coating is moreover advantageously homogeneous in terms of composition, on the surface of the core forming the microparticles.

Thus, according to a preferred embodiment variant, the coating arranged on the surface of the microparticles is obtained by spraying, in a fluidized bed, a solution or dispersion containing at least said polymers A and B on particles of active ingredient(s).

Preferably, the polymers A and B and if present the plasticizer(s) are sprayed in the solute state i.e. in a solubilized form in a solvent medium. This solvent medium generally contains organic solvents mixed or not mixed with water. The coating thus formed proves homogeneous in terms of composition as opposed to a coating formed by a dispersion of these same polymers, in a mostly aqueous liquid

According to a preferred embodiment variant, the sprayed solution contains less than 40% by weight water, in particular less than 30% by weight water and more particularly less than 25% by weight water, or even a water content less than or equal to 10% by weight relative to the total weight of the solvents.

This ability of the coating to preserve its physical integrity and its modified release properties are advantageously observed for coating levels varying from 3 to 85%, in particular from 5 to 60%, in particular from 10 to 50%, or even from 10 to 40%, and more particularly from 20 to 40% by weight of coating relative to the total weight of the microparticle.

The microparticles considered according to the invention possess an average diameter less than or equal to 2000 μm, in particular less than or equal to 1000 μm, in particular less than 800 μm, in particular less than 600 μm, or even less than 500 μm. The average diameter is determined by laser diffraction or sieve analysis according to the size scale to be characterized.

Generally, the use of the laser diffraction method, in particular as explained in the Pharmacopoeia 6th Edition, Chapter 2.9.31., to characterize a size by volume mean diameter, is preferred up to a size scale of 700 μm. As regards the characterization according to the sieve method, the choice of suitable sieve is clearly within the competence of a person skilled in the art who can refer to the European Pharmacopoeia 6th Edition, Chapter 2.9.38., proposing a method for estimating the granulometric distribution by sieve analysis.

Active Ingredients

The solid forms according to the invention are compatible with a wide range of active ingredients. For obvious reasons, their controlled and delayed release profile in terms of pH makes them quite particularly advantageous for active ingredients for which such release profiles are sought and therefore more particularly, the active ingredients for which it is sought to guarantee a significant release in the small intestine. This is essentially the case with pharmaceutical active ingredients.

Thus, the active ingredient contained in the coated microparticles according to the invention can be, for example, advantageously chosen from at least one of the following families of active ingredients: the anaesthetics, analgesics, antiasthmatics, allergy treatment agents, antineoplastics, anti-inflammatories, anticoagulants and antithrombotics, anti-convulsants, antiepileptics, antidiabetics, antiemetics, antiglaucoma agents, antihistaminics, anti-infective agents, in particular antibiotics, antifungals, antivirals, antiparkinsonians, anti-cholinergics, antitussives, carbonic anhydrase inhibitors, cardiovascular agents, in particular the lipopenics, anti-arrhythmic agents, vasodilators, anti-anginal drugs, anti-hypertensives, vasoprotectives and cholinesterase inhibitors, agents for treating disorders of the central nervous system, stimulants of the central nervous system, contraceptives, fertility promoters, dopamine receptor agonists, agents for the treatment of endometriosis, agents for treating gastrointestinal disorders, immunomodulators and immunosuppressors, agents for treating memory disorders, antimigraine drugs, myorelaxants, nucleoside analogues, agents for treating osteoporosis, parasympathomimetics, prostaglandins, psychotherapeutic agents such as sedatives, hypnotics, tranquillizers, neuroleptics, anxiolytics, psychostimulants and antidepressants, dermatological treatment agents, steroids and hormones, amphetamines, anorexigenics, non-analgesic pain relieving drugs, antiepileptics, barbiturates, benzodiazepines, hypnotics, laxatives, psychotropic drugs.

Certain of these families of active ingredients are in particular illustrated more particularly by the active ingredients utilized in the examples.

For obvious reasons, the particles according to the invention can be utilized for the purposes of determining active ingredients other than those identified above.

The solid form or solid composition according to the invention is advantageously presented in the form of a tablet, this tablet containing microparticles as defined above.

According to a particular embodiment, a solid form according to the invention has a load level of microparticles ranging from 5% to 60% by weight relative to its total weight, in particular 10% to 50% by weight, and more particularly 20 to 40% by weight.

Advantageously, the solid form containing the microparticles for modified release of the active ingredient also comprises standard physiologically acceptable excipients, which are useful for example for formulating the microparticles within a matrix and in particular in the form of a tablet.

These excipients can be in particular:
diluents
compression agents, such as microcrystalline cellulose or mannitol,
colorants,
disintegrators,
flow agents such as talc, colloidal silica,
lubricants such as for example glycerol behenate, stearates,
flavourings,
preservatives,
and mixtures thereof.

The choice of these excipients is clearly within the competence of a person skilled in the art.

According to a particular embodiment of the invention, the compression agents and/or diluents are in particular chosen from:
microcrystalline cellulose, such as for example the grades of Avicel® from FMC, the grades of Celphere® from Asahi Kasei, or powder cellulose,
calcium salts, such as calcium carbonate, phosphate and sulphate,
sugars, such as for example lactose, sucrose or sugar spheres,
mannitol, such as for example the grades of Pearlitol®, from Roquette, xylitol and erythritol.

A solid form according to the invention can in particular comprise one or more compression agent(s) and/or diluent(s) in a content ranging from 10% to 80% by weight, in particular 30% to 75% by weight, and more particularly 35% to 65% by weight relative to the total weight of the solid form.

According to another particular embodiment of the invention, the lubricants and/or flow agents are in particular chosen from:
the stearates, such as for example, magnesium stearate,
stearic acid,
glycerol behenate,
colloidal silica and
talc.

A solid form according to the invention can comprise one or more lubricant(s) and/or flow agent(s) in a content ranging from 0.1% to 5% by weight, in particular 0.5% to 2% by weight relative to the total weight of the solid form.

According to another particular embodiment of the invention, the binding agents are in particular chosen from:
the polymers derived from cellulose, such as hypromellose, methylcellulose, hydroxypropyl cellulose, hydroethylcellulose, ethylcellulose,
povidone, and
Poly(ethylene oxide).

The content of binding agent(s) in solid form according to the invention can range from 0% to 40% by weight, in particular 0% to 30% by weight, and more particularly 5 to 20% by weight relative to the total weight of the solid form.

According to a particular embodiment, a solid form according to the invention comprises, apart from the microparticles defined above, at least one compression agent and/or diluent, in particular chosen from microcrystalline cellulose, mannitol and mixtures thereof, and at least one lubricant and/or flow agent, in particular magnesium stearate and optionally at least one binding agent, in particular chosen from hypromellose and methylcellulose.

In particular, these different excipients are utilized at content levels as defined previously.

Other physiologically acceptable excipients can be added, in particular chosen from the disintegrators, colorants, flavourings and/or preservatives.

According to a particular embodiment, a solid form according to the invention comprises less than 1% by weight disintegrator(s) relative to its total weight, and more particularly, is free of disintegrant.

According to yet another particular embodiment, a solid form according to the invention is free of waxy compound which is insoluble in water, and in particular is free of waxes.

The final solid form, in the form of a tablet, can be coated according to the techniques and formulae known to a person skilled in the art in order to improve its presentation (colour, appearance, masking of taste, etc.).

The novel pharmaceutical forms according to the invention are original in their ability to manifest a controlled release profile and can be administered per os, in particular in a single, double or multiple daily dose.

Of course, a solid form according to the invention can combine different types of microparticles, which differ from each other for example with regard to the nature of the active ingredient they contain, and/or of the composition of the coating and/or the thickness of the coating.

According to a first embodiment, at least some of the modified release microparticles of the active ingredient each comprise a microparticle of the active ingredient, coated by at least one coating allowing the modified release of the active ingredient.

Preferably, the microparticle of the active ingredient is a granule comprising the active ingredient(s) and one or more physiologically acceptable excipients.

According to a second embodiment, at least part of the microparticles for modified release of the active ingredient each comprise a support particle, at least one active layer comprising the active ingredient(s) and coating the support particle, and at least one coating allowing the modified release of the active ingredient.

As specified previously, it can be also useful to mix in the same solid form, at least two types of microparticles with different release kinetics of the active ingredient, for example immediate release and modified release. It can also be useful to mix two (or more) types of microparticles, each containing a different active ingredient, released according to its own specific release profile.

A subject of the present invention is also a method for the preparation of a solid form for oral administration of at least one active ingredient, according to the invention comprising at least stages consisting of:

a) having microparticles formed wholly or partly by at least one active ingredient,

b) spraying in a fluidized bed onto the microparticles of stage a) a solution or dispersion containing at least one polymer A which is insoluble in the gastrointestinal fluids mixed with at least one polymer B possessing a solubilization pH value comprised within the pH range from 5 to 7, in a polymer(s) B/polymer(s) A weight ratio at least equal to 0.25,

c) mixing the microparticles of active ingredients, obtained at the end of stage b), with one or more physiologically acceptable excipients and capable of forming a matrix,

d) agglomerating the mixture formed in stage c) by compression.

According to an embodiment variant, the coated microparticles of active ingredients obtained at the end of stage c) can be mixed with other microparticles having different coating compositions and/or different sizes and/or particles of pure active ingredient prior to their transformation according to stage d).

The particles of active ingredients can be obtained beforehand according to several techniques such as for example:
extrusion/spheronization of the active ingredient, optionally with one or more physiologically acceptable excipient(s), and/or;
wet granulation of the active ingredient, optionally with one or more physiologically acceptable excipient(s), and/or;
compacting the active ingredient, optionally with one or more physiologically acceptable excipient(s), and/or;
spraying the active ingredient, optionally with one or more physiologically acceptable excipient(s), in dispersion or in solution in an aqueous or organic solvent on a support particle, and/or;
optionally sieved powder or crystals of the active ingredient;
the microparticles of the active ingredient may have been coated beforehand.

According to an embodiment variant, the solution in dispersion utilized in stage b) is a solution i.e. a solvent medium in which the polymers A and B are in the solute state.

Advantageously, it is a mixture of water and organic solvent(s) the water content of which is less than 40% by weight, in particular less than 30%, or even less than 25% by weight, in particular less than or equal to 10% by weight relative to the total weight of the mixture of solvents. The organic solvent can be chosen from the solvents known to a person skilled in the art. By way of example, the following solvents can be mentioned: acetone, isopropanol, ethanol and mixtures thereof.

The excipients capable of being combined in stage c) with microparticles of active ingredients can be diluents, binding agents, disintegrators, flow agents, lubricants, compounds which can modify the behaviour of the preparation in the digestive tract, colorants and/or flavouring.

These are useful general methodologies, which make it possible to produce the solid forms of the invention simply and economically.

The solid forms according to the present invention are advantageously obtained by compression. This compression can be carried out according to any conventional method and its implementation is clearly within the competence of a person skilled in the art.

Generally, all of the ingredients intended to form the matrix in which the microparticles are dispersed are mixed in the powdery state. These ingredients can moreover include one or more fillers, one or more lubricants, also in the powder state.

Once all of these ingredients and the particles have been mixed, by conventional methods, the resultant mixture is compressed in order to form the expected solid form and in particular a tablet.

The method for preparing such tablets is also well known to a person skilled in the art and is therefore not described in more detail hereafter.

These tablets, as mentioned previously, can if appropriate be subjected to complementary treatments aimed for example at forming on their surface a particular film-coating or coating intended to provide them with complementary properties or qualities.

The examples and figures which follow are presented by way of illustration and are non-limitative of the field of the invention.

### FIGURES

FIG. 1: Comparative in vitro release profiles obtained in a 0.1N HCl medium for tablets prepared according to Example 2 and microparticles prepared according to Example 1.

FIG. 2: Comparative in vitro release profiles obtained in a 0.05M potassium phosphate medium at a pH of 6.8 for tablets, prepared according to Example 2 and microparticles prepared according to Example 1.

FIG. 3: In vitro release profiles of microparticles of metformin prepared according to Example 3, obtained over 2 hours in the 0.1N HCl medium then in the 0.05M potassium phosphate medium at a pH of 6.8

FIG. 4: Comparative in vitro release profiles obtained in a 0.1N HCl medium for tablets and microparticles of metformin, not according to the invention, prepared according to Example 4.

FIG. 5: Comparative in vitro release profiles obtained in a 0.1N HCl medium for aciclovir tablets prepared according to Example 6 and aciclovir microparticles prepared according to Example 5.

FIG. 6: Comparative in vitro release profiles obtained in a 0.05M potassium phosphate medium at a pH of 6.8 for aciclovir tablets prepared according to Example 6 and aciclovir microparticles prepared according to Example 5.

FIG. 7: Comparative in vitro release profiles obtained in a 0.1N HCl medium comprising 0.2% by weight Cremophor RH 40®, for diclofenac tablets prepared according to Example 8 and diclofenac microparticles prepared according to Example 7.

FIG. 8: Comparative in vitro release profiles obtained in a 0.05M potassium phosphate medium at a pH of 6.8 for diclofenac tablets prepared according to Example 8 and diclofenac microparticles prepared according to Example 7.

FIG. 9: In vitro release profiles obtained in a 0.05 M phosphate medium at a pH of 6.8 and in a 0.1 N HCl medium for tablets prepared according to Example 9.

FIG. 10: Comparative in vitro release profiles, obtained for metformin tablets and microparticles, both prepared according to Example 10, during sequenced exposure to acid conditions (0.1N HCl medium) for 2 hours, then to a neutral pH (pH 6.8 medium).

In all of the figures, the symbol ♦ represents the tablet considered and the symbol X the corresponding microparticles and % D represents the percentage dissolved.

### EXAMPLE 1

### Preparation and Formulation of Microparticles of Metformin

Stage 1: Preparation of Granules (Coating Stage)

1800 g of metformin are introduced under stirring into a reactor which contains 2486 g of water. The solution is heated to 70° C. Once the metformin crystals have dissolved, the solution is sprayed onto 200 g of cellulose spheres (from Asahi Kasei) in a GPCG 1.1 fluidized bed in a bottom spray configuration (spraying the coating solution through a nozzle situated in the bottom part of the bed of particles).

After spraying, the product obtained is sieved on 200 μm and 800 μm sieves. 1888 g of granules ranging from 200 μm to 800 μm (which corresponds to the fraction of product having passed through the meshes of the 800 μm sieve and retained on the 200 μm sieve) are then recovered.

Stage 2: Coating Phase

490 g of granules obtained in stage 1 are coated at ambient temperature, in a GPCG 1.1 fluidized bed, with 105 g of a methacrylic acid and ethyl acrylate copolymer 1:1 (Eudragit L100-55 from Evonik), 84 g of cellulose acetate butyrate (from Eastman) and 21 g of triethylcitrate (from Morflex) dissolved in an acetone/water mixture (90/10 m/m). After spraying, the coated microparticles are recovered. Their volume mean diameter, determined by laser diffraction using a Mastersizer 2000 apparatus from Malvern Instruments equipped with the Scirocco 2000 dry route module, according to the calculation method “Adjusted standard analysis with normal sensitivity” (Model: General Purpose—normal sensitivity), is 631 μm.

### EXAMPLE 2

### Preparation of Tablets Containing the Microparticles of Example 1

4.0 g of the delayed and controlled release microparticles prepared in Example 1 are mixed with 4.0 g of hypromellose (Methocel E5 from Dow), 4.0 g of microcrystalline cellulose (Avicel PHI01 from FMC) and 0.2 g of magnesium stearate. This mixture is used in order to produce 800 mg tablets using a Perkin-Elmer hydraulic press.

In Vitro Dissolution Tests

The in vitro release kinetics of the tablets is monitored at 37±0.5° C. by UV spectrometry, on the one hand in 900 ml of a 0.1 N HCl medium and, on the other hand in 900 ml of a 0.05 M potassium phosphate medium at pH 6.8. The dissolution tests are carried out in a USP type II paddle apparatus. The speed of rotation of the paddles is 75 rpm.

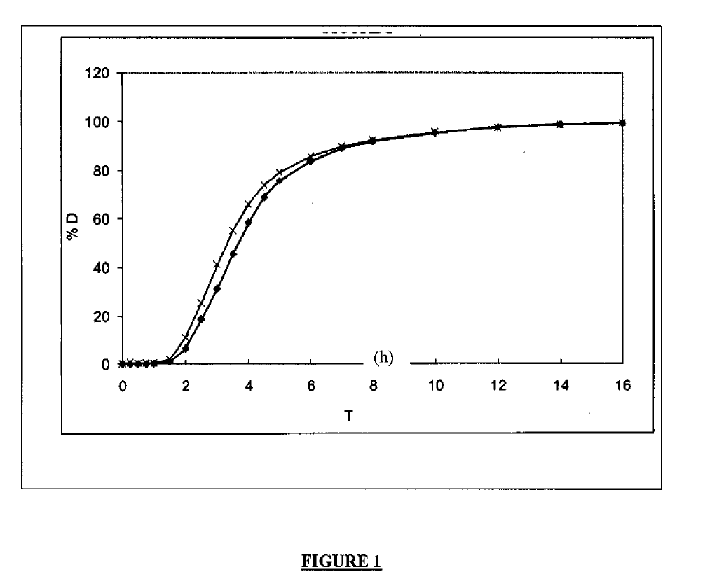

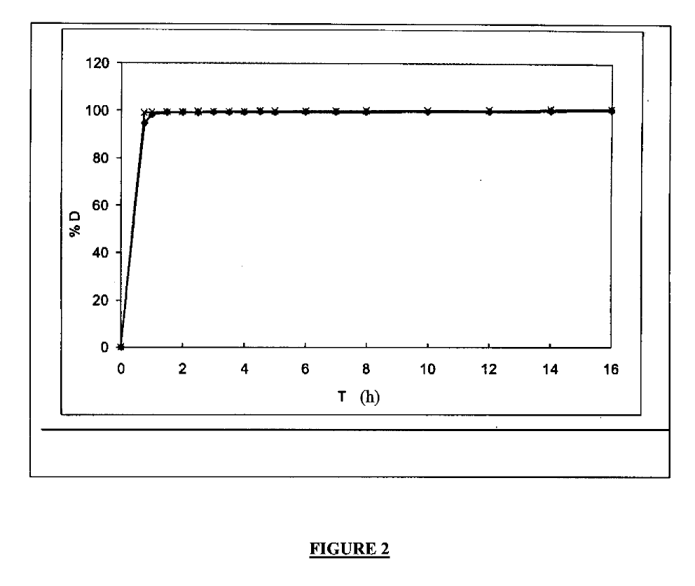

The results are illustrated in FIGS. 1 and 2 respectively. Each of FIGS. 1 and 2 also gives a comparative account of the release profile of the microparticles in a free form, i.e. according to those obtained in Example 1.

It is noted that the release profiles of the tablets and the microparticles in the free form are similar for each dissolution medium tested.

### EXAMPLE 3

### Coating of Metformin Crystals

Coating Phase

420 g of metformin crystals, sieved between 300 μm and 800 μm, are coated at ambient temperature, in a GPCG 1.1 fluidized bed, with 165 g of a methacrylic acid and ethyl acrylate copolymer 1:1 (Eudragit L100-55 from Evonik), 132 g of cellulose acetate butyrate (from Eastman) and 33 g of triethylcitrate (from Morflex) dissolved in an acetone/water mixture (90/10 m/m). At the end of the spraying, the expected microparticles are recovered. Their volume mean diameter, determined by laser diffraction using a Mastersizer 2000 apparatus from Malvern Instruments equipped with the Scirocco 2000 dry route module, according to the calculation method “Adjusted standard analysis with normal sensitivity” (Model: General Purpose—normal sensitivity), is 495 μm.

Dissolution Profiles Under Sequential Exposure Conditions

The in vitro kinetics of the microparticles prepared above is monitored at 37° C.±0.5° C. by UV spectrometry for 2 hours in a 0.1 N HCl medium then, after adjustment of the pH, in a 0.05 M potassium phosphate medium at pH 6.8. The dissolution test is carried out in a USP type II paddle apparatus, in 900 ml of medium. The speed of rotation of the paddles is 75 rpm.

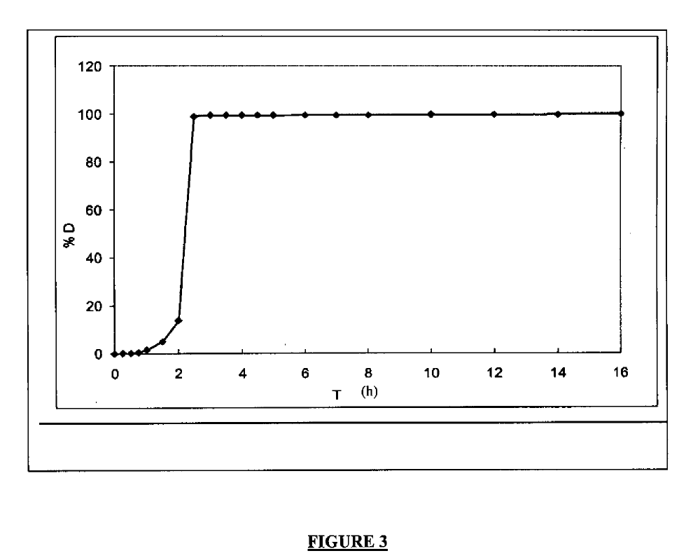

The dissolution profile is presented in FIG. 3.

### EXAMPLE 4

### Preparation and Formulation of Microparticles not According to the Invention in a Tablet

Phase 1: Preparation of the Granules (Coating Stage)

1746 g of metformin and 54 g of povidone (Plasdone K29/32 from ISP) are introduced under stirring into a reactor containing 2486 g of water. The solution is heated to 74° C. When the metformin and povidone crystals are dissolved, the solution is sprayed onto 450 g of cellulose spheres (from Asahi Kasei) in a GPCG 1.1 fluidized bed in a bottom spray configuration. 2224 g of metformin granules are obtained.

Phase 2: Coating Phase

455 g of granules, as prepared above, are coated in a GPCG 1.1 fluidized bed, with 117 g of a methacrylic acid and ethyl acrylate copolymer 1:1 (Eudragit L100-55 from Evonik) and 78 g of hydrogenated cotton seed oil (Lubritab® from JRS Pharma), dissolved in 1305 g of isopropanol at 78° C. After spraying, the product is heated at 55° C. for 2 hours. 638 g of microparticles are obtained.

Preparation of Tablets

4.0 g of microparticles, as prepared above, are mixed with 3.0 g of hypromellose (Methocel E5 from Dow), 3.0 g of microcrystalline cellulose (Avicel PH101 from FMC), 2.0 g of mannitol (Pearlitol SD 200 from Roquette) and 0.2 g of magnesium stearate. This mixture is used in order to produce 800 mg tablets using a Perkin-Elmer hydraulic press.

In Vitro Dissolution Tests

The in vitro release kinetics of the metformin microparticles and tablets prepared as described previously are monitored at 37±0.5° C. by UV spectrometry in 900 ml of 0.1 N HCl medium. The dissolution tests are carried out in a USP type II paddle apparatus. The speed of rotation of the paddles is 75 rpm.

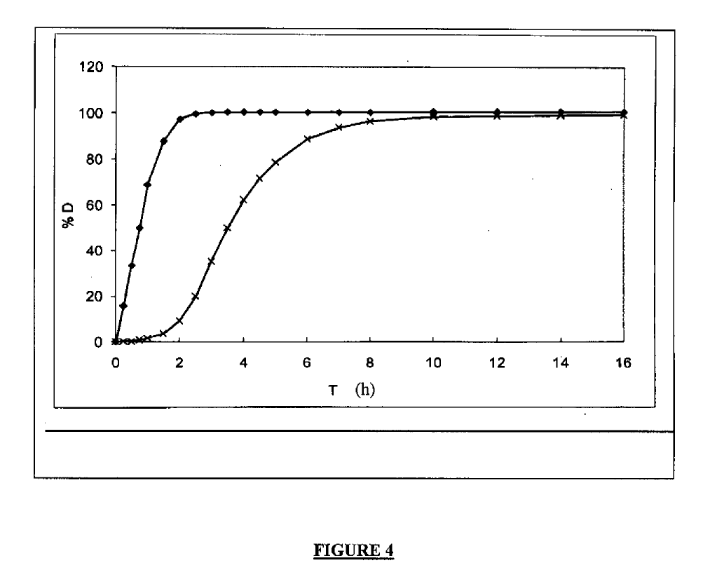

The dissolution profiles are illustrated in FIG. 4. It is noted that the release profiles of the tablets and the microparticles in the free form are different. The release profile of the tablet does not correspond to that of the microparticles. It is more rapid, revealing a lack of control.

### EXAMPLE 5

### Production of Aciclovir Microparticles

Phase 1: Preparation of the Granules (Coating Stage)

810 g of aciclovir and 90 g of povidone (Plasdone K29/32 from ISP) are introduced under stirring into 2100 g of water. When the aciclovir crystals and the povidone are dissolved, the solution is sprayed onto 600 g of cellulose spheres (from Asahi Kasei) in a GPCG 1.1 fluidized bed in a bottom spray configuration. After coating, the product is sieved on sieves with a mesh size of 200 μm and 800 μm. Aciclovir granules ranging from 200 μm to 800 μm, corresponding to the fraction of product passed through the meshes of the 800 μm sieve and recovered on the 200 μm sieve, are obtained.

Phase 2: Coating Phase

350 g of granules, as prepared above, are coated at ambient temperature in a GPCG 1.1 fluidized bed, with 45 g of a methacrylic acid and ethyl acrylate copolymer 1:1 (Eudragit L100-55 from Evonik), 90 g of a type A ammonio (meth)acrylate copolymer (Eudragit RL100 from Evonik) and 15 g of dibutylphthalate (from Merck), dissolved in an acetone/water mixture (90/10 m/m). After spraying, microparticles are obtained. The volume mean diameter of the coated aciclovir microparticles, determined by laser diffraction on a Mastersizer 2000 apparatus from Malvern Instruments equipped with the Scirocco 2000 dry route module, according to the calculation method “Adjusted standard analysis with normal sensitivity” (Model: General Purpose—normal sensitivity), is 412 μm.

### EXAMPLE 6

### Dissolution in 0.1N HCl and at a pH of 6.8 of Tablets Comprising Aciclovir Microparticles

2.0 g of delayed and controlled release microparticles, such as those prepared in Example 5, are mixed with 1.0 g of hypromellose (Methocel E5 from Dow), 2.0 g of microcrystalline cellulose (Avicel PH101 from FMC) and 1.0 g of mannitol (Pearlitol SD200 from Roquette) and 0.1 g of magnesium stearate. This mixture is used for the production of tablets weighing 800 mg.

The in vitro release kinetics of the tablets are monitored at 37±0.5° C. by UV spectrometry, on the one hand in 900 ml of a 0.1 N HCl medium and on the other hand in 900 ml of a 0.05M potassium phosphate medium at a pH of 6.8. The dissolution tests are carried out in a USP type II paddle apparatus. The speed of rotation of the paddles is 75 rpm.

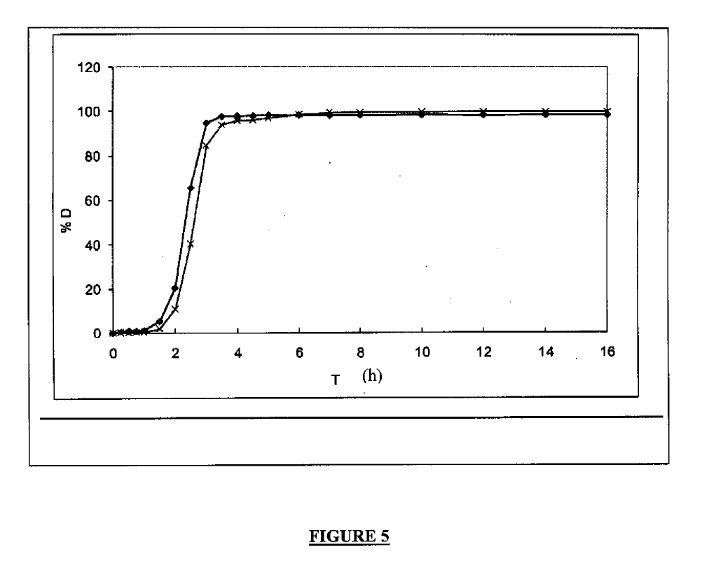

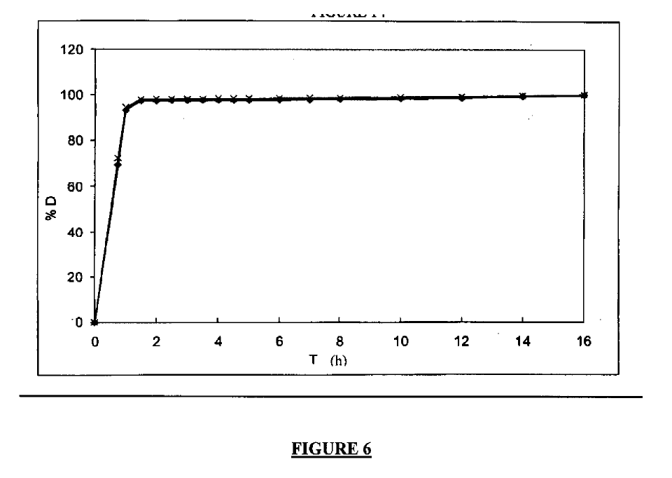

The dissolution profiles of the tablets are compared in FIGS. 5 and 6 with the dissolution profiles of the aciclovir microparticles prepared in Example 5.

The release profiles of the tablets and the microparticles in the free form are identical for each dissolution medium tested.

### EXAMPLE 7

### Production of Diclofenac Microparticles

Phase 1: Preparation of Granules (Coating Stage)

100 g of sodium diclofenac and 400 g of povidone (Plasdone K29/32 from ISP) are introduced under stirring into 1674 g of water. The solution is heated to 70° C. After complete dissolution of the ingredients, the solution is sprayed onto 600 g of cellulose spheres (from Asahi Kasei) in a GPCG 1.1 fluidized bed in a bottom spray configuration. The granules obtained are sieved on 200 μm to 500 μm sieves. Diclofenac granules ranging from 200 μm to 500 μm are obtained.

Phase 2: Coating Phase

420 g of granules as prepared above are coated at ambient temperature in a GPCG 1.1 fluidized bed, with a solution comprising 108 g of a methacrylic acid and ethyl acrylate copolymer 1:1 (Eudragit L100-55 from Evonik), 54 g of a type B ammonio (meth)acrylate copolymer (Eudragit RS100 from Evonik), 18 g of triethylcitrate (of Morflex), dissolved in an acetone/water mixture (95/5 m/m). After spraying, the product is sieved on 630 μm. The microparticles thus obtained have a volume mean diameter, determined by laser diffraction using a Mastersizer 2000 apparatus from Malvern Instruments equipped with the Scirocco 2000 dry route module, according to the calculation method “Adjusted standard analysis with normal sensitivity” (Model: General Purpose—normal sensitivity), of 411 μm.

### EXAMPLE 8

### Dissolution of Tablets Comprising Diclofenac Microparticles

112.5 g of delayed and controlled release microparticles, such as those prepared in Example 7, are mixed with 157.8 g of microcrystalline cellulose (Avicel PHI01 from FMC), 28.2 g of mannitol (Pearlitol SD200 from Roquette) and 1.5 g of magnesium stearate. This mixture is used for the production of round 700 mg tablets with a diameter of 12 mm, using a Korsch XP1 press. The compressive force applied to the mixture is 15 kN. The tablets thus produced have a hardness of approximately 98 N.

The in vitro release kinetics of the above tablets are monitored at 37±0.5° C. by UV spectrometry, on the one hand, in 900 ml of a 0.1 N HCl medium containing 0.2% by mass of Cremophor RH 40, and, on the other hand, in 900 ml of a 0.05M potassium phosphate medium at pH 6.8. The dissolution tests are carried out in a USP type II paddle apparatus. The speed of rotation of the paddles is 75 rpm.

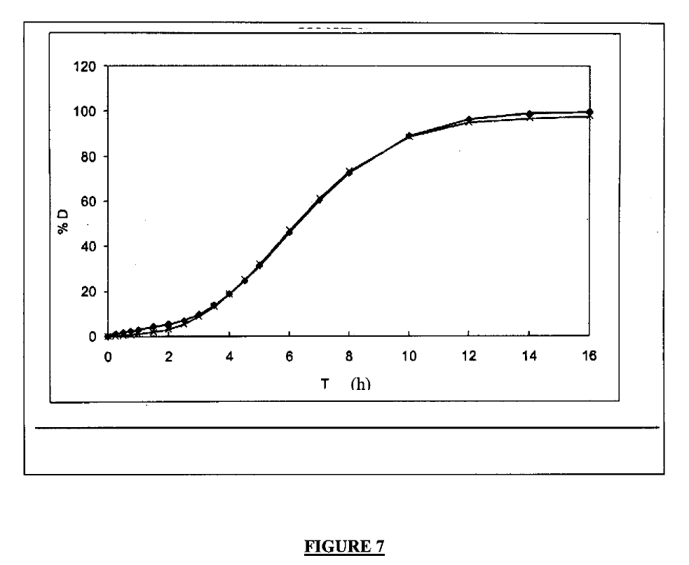

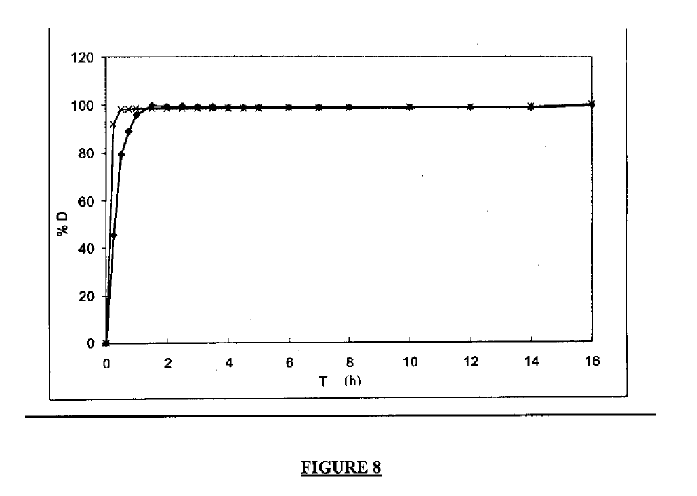

The dissolution profiles of the tablets are compared in FIGS. 7 and 8 with the dissolution profiles of the diclofenac microparticles prepared in Example 7.

The release profiles of the tablets and the microparticles in the free form are identical for each dissolution medium tested.

### EXAMPLE 9

### Preparation of Tablets Containing the Non-Coated Granules and the Microparticles of Example 1

1.0 g of non-coated granules prepared in Example 1 and 3.0 g of the delayed and controlled release microparticles prepared in Example 1, are mixed with 5.0 g of microcrystalline cellulose (Avicel PH101 from FMC), 0.9 g of mannitol (Pearlitol SD 100 from Roquette) and 0.1 g of magnesium stearate. This mixture is used in order to produce 800 mg tablets using a Perkin-Elmer hydraulic press.

In Vitro Dissolution Tests

The in vitro release kinetics of the tablets are monitored at 37±0.5° C. by UV spectrometry, on the one hand, in 900 ml of a 0.1 N HCl medium and, on the other hand, in 900 ml of a 0.05M potassium phosphate medium at pH 6.8. The dissolution tests are carried out in a USP type II paddle apparatus. The speed of rotation of the paddles is 75 rpm.

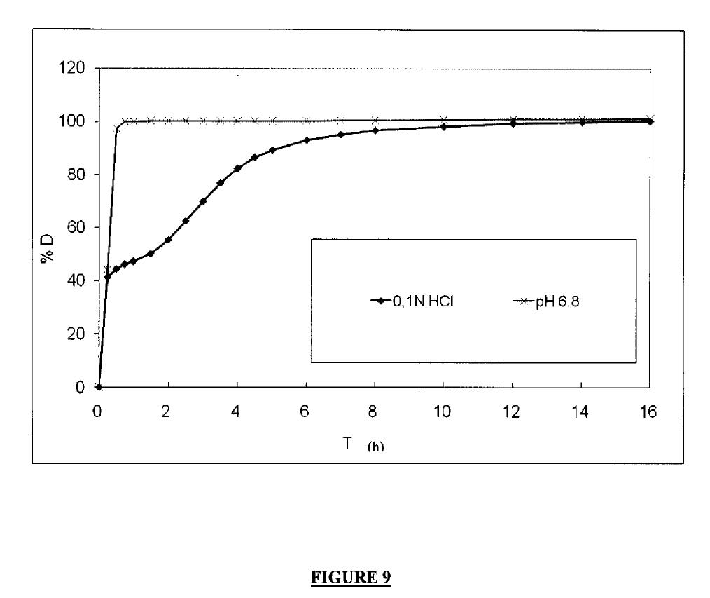

The test results are presented in FIG. 9.

In the 0.1N HCl medium, the fraction of active ingredient released immediately corresponds to the fraction of active ingredient contained in the non-coated granules used for the production of the tablets.

### EXAMPLE 10

### Preparation of Metformin Microparticles and Tablets

Coating Phase

420 g of granules obtained in stage 1 of Example 1 are coated at ambient temperature in a GPCG 1.1 fluidized bed with 37 g of a methacrylic acid and ethyl acrylate copolymer (Eudragit L100-55 from Evonik), 29.6 g of ethyl cellulose (Ethocel 20 premium from Dow) and 7.4 g of triethylcitrate (from Morflex) dissolved in an acetone/water mixture (90/10 m/m). After spraying, the coated microparticles are recovered. Their volume mean diameter, determined by laser diffraction using a Mastersizer 2000 apparatus from Malvern Instruments equipped with the Scirocco 2000 dry route module is 640 μm.

Preparation of the Tablets

2.0 g of delayed and controlled release microparticles prepared in the previous stage are mixed with 2.0 g of hypromellose (Methocel E5 from Colorcon), 3.0 g of microcrystalline cellulose (Avicel PH101 from FMC), 3.0 g of mannitol (Perlitol SD 200 from Roquette) and 0.2 g of magnesium stearate. This mixture is used to produce 800 mg tablets using a Perkin-Elmer hydraulic press.

Note: It is also possible to obtain, in a similar manner, compounds containing microparticles prepared as described above, but replacing the 37 g of Eudragit L100-55 with a mixture of 14.8 g of Eudragit L100-55 and 22.2 g of Eudragit S100.

Dissolution Profiles Under Sequential Exposure Conditions

The in vitro dissolution profiles of the tablets and microparticles prepared above are monitored at 37±0.5° C. by UV spectrometry in 900 ml of 0.1 N HCl for 2 hours then, after adjustment of the pH and the salinity of the medium, at pH 6.8 and 0.05 M of potassium phosphate. The dissolution test is carried out in a USP type II paddle apparatus. The speed of rotation of the paddles is 75 rpm.

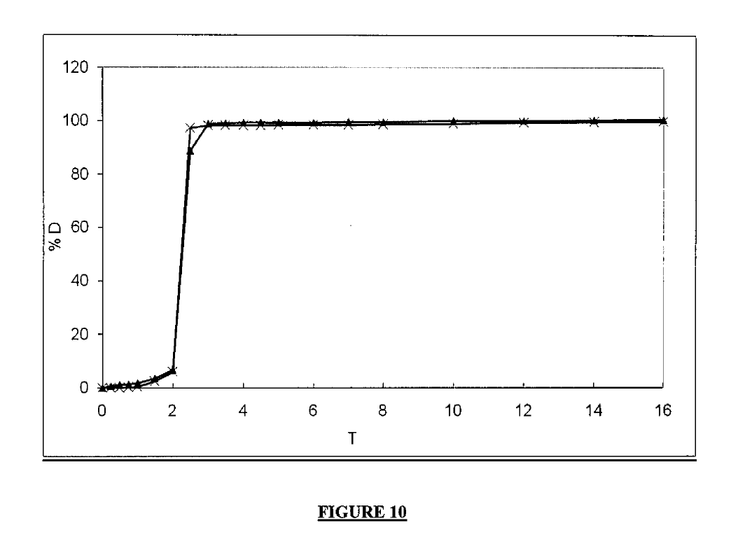

The dissolution profiles obtained for the microparticles and the tablets are compared in FIG. 10.

1. Solid form, intended for the administration by oral route of at least one active ingredient and capable of guaranteeing a double release mechanism of said active ingredient, the first being determined by time and the second being determined by pH, characterized in that said active ingredient is present there in the form of a microparticle system the microparticles of which possess a core formed wholly or partly by said active ingredient and coated with at least one layer determining said release profile of said active ingredient and formed by a material made up of at least:
25 to 75% by weight relative to the total weight of said coating of at least one polymer A which is insoluble in the gastro-intestinal fluids,
25 to 75% by weight relative to the total weight of said coating of at least one polymer B possessing a solubilization pH value comprised within the pH range from 5 to 7, and
0 to 25% by weight relative to the total weight of said coating of at least one plasticizer,
said polymers A and B being present in a polymer(s) B/polymer(s) A weight ratio at least equal to 0.25.

2. Solid form according to claim 1, possessing a modified three-phase release profile.

3. Solid form according to claim 1 or 2, characterized in that it is presented in a matrix form within which said microparticles are dispersed.

4. Solid form according to claim 1, 2 or 3, characterized in that it is a tablet.

5. Solid form according to any one of the previous claims, in which the polymer A is chosen from ethylcellulose, cellulose acetate butyrate, cellulose acetate, type “A” or type “B” ammonio (meth)acrylate copolymers, poly(meth)acrylic acid esters, and mixtures thereof.

6. Solid form according to any one of the previous claims, in which the coating of the microparticles contains 25% to 60% by weight, in particular 25 to 55% by weight and more particularly 30 to 50% of polymer(s) A relative to its total weight.

7. Solid form according to any one of the previous claims, in which the polymer B is chosen from the methacrylic acid and methyl methacrylate copolymer(s), the methacrylic acid and ethyl acrylate copolymer(s), cellulosic derivatives such as cellulose acetate phthalate, cellulose acetate succinate, cellulose acetate trimellilate, hydroxypropylmethylcellulose phthalate, hydroxypropylmethylcellulose acetate succinate, shellac gum, polyvinyl acetate phthalate, and mixtures thereof.

8. Solid form according to any one of the previous claims, in which the coating of the microparticles contains 30to 75% by weight, in particular 35 to 70% by weight, in particular 40% to 60% by weight of polymer(s) B relative to its total weight.

9. Solid form according to any one of the previous claims, in which the coating of the microparticles is formed by at least one mixture of ethylcellulose, cellulose acetate butyrate or type “A” or “B” ammonio (meth)acrylate copolymer with at least one methacrylic acid and ethyl acrylate copolymer or a methacrylic acid and methyl methacrylate copolymer or a mixture thereof.

10. Solid form according to any one of the previous claims, in which the coating comprises the polymers A and B in a polymer(s) B/polymer(s) A weight ratio greater than or equal to 0.3, in particular greater than or equal to 0.4, in particular greater than or equal to 0.5, or even advantageously greater than or equal to 0.75.

11. Solid form according to any one of the previous claims, in which the coating of the microparticles comprises moreover at least one plasticizer.

12. Solid form according to the previous claim, in which the coating of the microparticles comprises less than 25% by weight, in particular 1% to 20% by weight, and more preferably 5% to 20% by weight of plasticizer(s) relative to its total weight.

13. Solid form according to any one of the previous claims, in which the coating of the microparticles is composed of a single layer formed by said material.

14. Solid form according to any one of the previous claims, in which the coating arranged on the surface of the microparticles is present at a coating level varying from 3 to 85% by weight, in particular 5 to 60% by weight, in particular 10 to 50%, or even 10to 40%, and more particularly 20 to 40% by weight of coating, relative to the total weight of said microparticle.

15. Solid form according to any one of the previous claims, in which the coating arranged on the surface of the microparticles is obtained by the spraying in a fluidized bed, of a solution containing at least said polymers A and B in the solute state onto particles of active ingredient(s).

16. Solid form according to any one of the previous claims, in which the microparticles possess an average diameter less than or equal to 2000 μm, in particular less than or equal to 1000 μm, in particular less than 800 μm, in particular less than 600 μm, or even less than 500 μm.

17. Solid form according to any one of the previous claims, in which the active ingredient is chosen from the anaesthetics, analgesics, antiasthmatics, allergy treatment agents, the antineoplastics, anti-inflammatories, anticoagulants and antithrombotics, anti-convulsants, antiepileptics, antidiabetics, antiemetics, antiglaucoma agents, antihistaminics, anti-infective agents, in particular antibiotics, antifungals, antivirals, antiparkinsonians, anti-cholinergics, antitussives, carbonic anhydrase inhibitors, cardiovascular agents, in particular the lipopenics, anti-arrhythmic agents, vasodilators, anti-anginal drugs, anti-hypertensives, vasoprotectives and cholinesterase inhibitors, agents for treating disorders of the central nervous system, stimulants of the central nervous system, contraceptives, fertility promoters, dopamine receptor agonists, agents for the treatment of endometriosis, agents for treating gastro-intestinal disorders, immunomodulators and immunosuppressors, agents for treating memory disorders, antimigraine drugs, myorelaxants, nucleoside analogues, agents for treating osteoporosis, parasympathomimetics, prostaglandins, psychotherapeutic agents such as sedatives, hypnotics, tranquillizers, neuroleptics, anxiolytics, psychostimulants and antidepressants, dermatological treatment agents, steroids and hormones, amphetamines, anorexigenics, non-analgesic pain relieving drugs, antiepileptics, barbiturates, benzodiazepines, hypnotics, laxatives, psychotropic drugs.

18. Solid form according to any one of the previous claims, comprising at least two types of microparticles differing from each other by distinct release profiles.

19. Solid form according to any one of the previous claims, comprising at least two types of microparticles, in which said types differ from each other at least by the nature of the active ingredient that they contain and/or by the composition of their coating and/or the thickness of their coating.

20. Method for the preparation of a solid form for the oral administration of at least one active ingredient according to any one of the previous claims, comprising at least stages involving:
a) having microparticles formed wholly or partly by at least one active ingredient,
b) spraying in a fluidized bed onto the microparticles of stage a), a solution or dispersion containing at least one polymer A which is insoluble in the gastrointestinal fluids mixed with at least one polymer B possessing a solubilization pH value comprised within the pH range from 5 to 7, in a polymer(s) B/polymer(s) A weight ratio at least equal to 0.25,
c) mixing the microparticles of coated active ingredients obtained at the end of stage b) with one or more physiologically acceptable excipients capable of forming a matrix
d) agglomerating the mixture formed in stage c) by compression.

21. Method according to the previous claim, in which the polymers A and B are as defined in claims 5 to 10.

22. Method according to claim 20 or 21 in which the microparticles obtained at the end of stage b) are as defined in claims 11 to 16.

23. Microparticles possessing a core formed wholly or partly by at least one active ingredient, said core being coated with at least one layer determining a double release mechanism of said active ingredient, the first being determined by time and the second being determined by the pH, and formed by a material made up of at least:
25 to 75% by weight, in particular 25% to 60% and, still more preferably, 25 to 55% by weight, and still more particularly 30 to 50% relative to the total weight of said coating of at least one polymer A which is insoluble in the gastro-intestinal fluids and chosen from ethylcellulose, cellulose acetate butyrate, a type “A” or type “B” ammonio (meth)acrylate copolymer, poly(meth)acrylic acid esters and mixtures thereof, and
25 to 75% by weight, in particular 30 to 75%, in particular 35 to 70%, or even 40 to 60% by weight relative to the total weight of said coating of at least one polymer B possessing a solubilization pH value comprised within the pH range varying from 5 to 7 and chosen from a methacrylic acid and methyl methacrylate copolymer, a methacrylic acid and ethyl acrylate copolymer and mixtures thereof.

24. Microparticles according to the previous claim, the coating of which is formed by at least one polymer B/polymer A pair chosen from the following pairs:
methacrylic acid and ethyl acrylate, 1:1 copolymer/ethylcellulose,
methacrylic acid and methyl methacrylate 1:2 copolymer/ethylcellulose,
mixture of methacrylic acid and ethyl acrylate 1:1 copolymer and methacrylic acid and methyl methacrylate, 1:2 copolymer/ethylcellulose,
methacrylic acid and ethyl acrylate 1:1 copolymer/cellulose acetate butyrate,
methacrylic acid and methyl methacrylate 1:2 copolymer/cellulose acetate butyrate,
mixture of methacrylic acid and ethyl acrylate 1:1 copolymer and methacrylic acid and methyl methacrylate 1:2 copolymer/cellulose acetate butyrate,
methacrylic acid and ethyl acrylate copolymer 1:1 copolymer/type “A” or type “B” ammonio (meth)acrylate,
methacrylic acid and methyl methacrylate 1:2 copolymer/type “A” or type “B” ammonio (meth)acrylate copolymer,
mixture of methacrylic acid and ethyl acrylate 1:1 copolymer and methacrylic acid and methyl methacrylate 1:2 copolymer/type “A” or type “B” ammonio (meth)acrylate copolymer.
